(19) United States
(12) Patent Application Publication
(10) Pub. No.: US 2025/0387489 A1
(43) Pub. Date: Dec. 25, 2025

(54) METHOD FOR PROVIDING A TOPICAL FORMULATION BASE

(71) Applicant: Peep Club Ltd, London (GB)

(72) Inventor: Nicola ALEXANDER, London (GB)

(21) Appl. No.: 19/181,140

(22) Filed: Apr. 16, 2025

(30) Foreign Application Priority Data
May 20, 2024 (GB) 2407163.1

(51) Int. Cl. A61K 47/44; A61K 9/00

(52) U.S. Cl. CPC A61K 47/44; A61K 9/0014

(57) ABSTRACT

Disclosed is a method for providing a topical formulation base. The method includes providing water, salt, and ozonated sea buckthorn oil, and combining the water, salt, and ozonated sea buckthorn oil. Also disclosed are methods for providing a topical formulation, topical formulation base compositions, topical formulation compositions, non-therapeutic methods of using said compositions, and compositions for use in therapy.

### CROSS-REFERENCE TO RELATED APPLICATIONS

This application claims priority to GB Patent Application No. 2407163.1, filed May 20, 2024, under 35 U.S.C. § 119 (a). The above-referenced patent application is incorporated by reference in its entirety.

### BACKGROUND OF THE INVENTION

### Field of the Invention

The present invention relates to methods for providing a topical formulation base, methods for providing a topical formulation, topical formulation base compositions, topical formulation compositions, non-therapeutic methods of using said compositions, and compositions for use in therapy.

### Description of the Related Technology

A topical formulation base is a composition to which further components may be added to provide a topical formulation. Typically, different further components may be added to a topical formulation base to provide different topical formulations. That is, a plurality of topical formulations having different compositions and/or characteristics can be obtained from the same topical formulation base. A topical formulation base may include components which impart advantageous or desirable qualities to a topical formulation comprising the topical formulation base. In some instances, a topical formulation base is itself suitable for use as a topical formulation.

Topical formulations are compositions to be applied to the skin of a user, and are often applied by hand. In applying a formulation to the skin, bacteria and/or fungus can be transferred to the skin of the user, thereby increasing the risk of the user contracting a bacterial or fungal infection. The eye and the area surrounding the eye, typically referred to as the periorbital region, is particularly susceptible to bacterial or fungal infection through topical application of formulations to the skin.

There is a need for topical formulations, such as cosmetic topical formulations, to have good stability properties to allow for an acceptable shelf-life. Topical formulations often include preservatives, fragrances, alcohols, emulsifiers, and surfactants to improve stability, shelf life, and smell.

There is a desire to provide compositions for topical application, and methods of providing said compositions, which avoid or reduce the risk of infection while retaining acceptable stability characteristics.

### SUMMARY

According to a first aspect, there is provided a method for providing a topical formulation base, the method comprising providing water, salt, and ozonated sea buckthorn oil, and combining the water, salt, and ozonated sea buckthorn oil.

In examples, the providing the ozonated sea buckthorn oil comprises providing ozone gas to sea buckthorn oil. Suitably, ozonated sea buckthorn oil may be referred to ozonized sea buckthorn oil. Further, providing ozone gas to sea buckthorn oil to provide ozonated sea buckthorn oil may be referred to herein as ozonation of sea buckthorn oil.

Sea buckthorn oil comprises a relatively high proportion of polyunsaturated fatty acids (Omega 3s and Omega 6s) and monounsaturated fatty acids (Omega 7s and Omega 9s) compared with some other vegetable oils. Without wishing to be bound by theory, the inventor considers that the ratio of fatty acids in sea buckthorn oil makes it particularly suitable for ozonation compared with other vegetable oils. Moreover, ozonation of sea buckthorn oil may cause various peroxidation reactions of the fatty acids present in the sea buckthorn oil, such that ozonated sea buckthorn oil is chemically distinct from sea buckthorn oil that has not undergone an ozonation process. In examples, ozonated sea buckthorn oil comprises a higher proportion of peroxides, hydroperoxides, and ozonides compared with sea buckthorn oil that has not been subjected to ozone treatment.

Surprisingly, the inventor has identified that, upon ozonation, the above-recited oils may provide an anti-microbial and/or anti-fungal effect when applied topically. In particular, the inventor has identified that ozonated sea buckthorn oil may have improved anti-microbial and/or anti-fungal compared with non-ozonated sea buckthorn oil.

Further, the inventor has identified that ozonated sea buckthorn oil may impart improved moisturisation, skin tolerance, skin calming, and skin redness reduction properties to the topical formulation base compared with sea buckthorn oil that has not been treated with ozone (also referred to herein as “non-ozonated sea buckthorn oil”).

Further still, the inventor has identified that ozonating the sea buckthorn oil according to examples described herein allows for the provision of topical formulation bases and topical formulation having improved stability and shelf-life, e.g. shelf-life of at least 12 months. In particular, this method may obviate the need to include preservatives, fragrances, alcohols, emulsifiers, and surfactants to improve stability, shelf life, and smell. The inventor has identified that such preservatives, fragrances, alcohols, emulsifiers, and surfactants may act as irritants to the skin of a user, particularly when applied to the face, more particularly to the area of the face surrounding the eyes. Accordingly, obviating the need to include preservatives, fragrances, alcohols, emulsifiers, and surfactants surprisingly allows for the provision of a topical formulation which is less likely to irritate the skin of a user.

In examples, the providing ozonated sea buckthorn oil comprises providing sea buckthorn oil in a first vessel and providing ozone gas to the sea buckthorn oil in the first vessel.

In examples, the ozone gas is provided to the sea buckthorn oil in the first vessel until the concentration of ozone in the first vessel is at least 15 grammes per cubic metre (g/m³), such as at least 25 g/m³. Advantageously, the inventor has identified that supplying ozone gas to at least this concentration provides ozonated sea buckthorn oil having characteristics suitable for use in a topical formulation base.

In examples, the pressure within the first vessel is atmospheric pressure for at least 50% of the duration of providing the ozone gas to the sea buckthorn oil, such as at least 80% or 90%, or substantially all of the duration of providing the ozone gas to the sea buckthorn oil.

In examples, the ozone gas is provided to the sea buckthorn oil for a minimum duration. For example, the duration of providing the ozone gas is at least 5 hours, preferably at least 12 hours, more preferably at least 24 hours, or still more preferably at least 48 hours. Typically, the duration of providing the ozone gas referred to above is continuous, e.g. providing the ozone gas to the sea buckthorn oil continuously for at least 5 hours, preferably at least 12 hours, more preferably at least 24 hours, or still more preferably at least 48 hours.

In examples, the ozone gas is provided to the sea buckthorn oil at or above a minimum flow rate. For example, the ozone gas is provided to the sea buckthorn oil at a flow rate of at least 100 litres per hour (L/h), such as at least 150 L/h, preferably at least 200 L/h. Typically, the ozone gas is provided to the sea buckthorn oil at or above the minimum flow rate for at least 50% of the duration of providing the ozone gas to the sea buckthorn oil, such as at least 80%, or 90%, or substantially all of the duration of providing the ozone gas to the sea buckthorn oil.

Typically, the degree of ozonation of the sea buckthorn oil is proportional parameters of the ozonation process, such as the time of contact between the ozone gas and the sea buckthorn oil, e.g. the duration of continuous provision of ozone gas to the sea buckthorn oil, and the flow rate of ozone gas provided to the sea buckthorn oil.

Advantageously, the inventor has identified that ozonation of sea buckthorn oil for at least 5 hours at a flow rate of at least 100 L/h may provide a composition having a stable shelf-life of at least six months. Further, ozonation of sea buckthorn oil for at least 24 hours at a flow rate of at least 100 L/h may provide a composition having a stable shelf-life of at least twelve months.

In examples, the ozone gas is provided to the sea buckthorn oil by bubbling the ozone gas through the sea buckthorn oil. For example, sea buckthorn oil is provided in the first vessel, and ozone is bubbled through the sea buckthorn oil in the first vessel. Advantageously, providing the ozone by bubbling it through the sea buckthorn oil may allow for improved ozonation rates/efficiency. In examples, the ozone is continuously bubbled through the sea buckthorn oil for the duration of the providing the ozone.

In examples, the ozone gas is provided to the sea buckthorn oil in the absence of water; the first vessel is essentially free of water.

In examples, the ozone gas is provided with an ozone generator. For example, the first vessel is in (optionally selective) fluid communication with the ozone generator, ozone is generated by the ozone generator, and the generated ozone is supplied to the sea buckthorn oil. In some examples, the ozone generator generates ozone from atmospheric air, and provides a fluid comprising ozone to the first vessel. In these examples, the fluid which is provided from the ozone generator to the first vessel typically comprises appreciable amounts of components other than ozone, such as nitrogen, which in the context of the present disclosure may be considered an impurity. In other, preferred, examples, the ozone generator generates ozone from an oxygen source. For example, the ozone generator is in (optionally selective) fluid communication with an oxygen source (such as an oxygen concentrator or an oxygen bottle), generates ozone from essentially pure oxygen received from the oxygen source, and provides a fluid comprising, or consisting essentially of, ozone to the first vessel. Advantageously, providing ozone to the sea buckthorn oil with an oxygen source and ozone generator may provide an ozonated sea buckthorn oil having a lower proportion of impurities.

Suitably, the degree of ozonation of the ozonated sea buckthorn oil may be expressed by the peroxide value (PV) of the ozonated sea buckthorn oil. The peroxide value refers to the amount of peroxide oxygen per kilogram of the oil. PV may be expressed in milliequivalents of active oxygen per kilogram (mEq/kg), but may also be expressed as millimoles of active oxygen per kilogram (mmol/kg). The value expressed in mmol/kg is half that expressed in mEq/kg. PV may be determined according to ISO standard ISO 3960:2017, such that the peroxide value indicates the quantity of those substances in the oil, expressed in terms of active oxygen, that oxidize potassium iodide under the conditions specified in the standard.

In examples, the ozone gas is provided to the sea buckthorn oil to provide ozonated sea buckthorn oil having a peroxide value equal to or greater than 300 mEq/kg, preferably equal to or greater than 600 mEq/kg, more preferably greater than or equal to 800 mEq/kg, still more preferably greater than or equal to 1,500 mEq/kg. In examples, the ozonated sea buckthorn oil has a peroxide value equal to or less than 2,200 mEq/kg.

Advantageously, the inventor has identified that ozonated sea buckthorn oil having a peroxide value equal to or greater than 300 mEq/kg may provide a product having a stable shelf-life of at least six months. Further, ozonated sea buckthorn oil having a peroxide value equal to or greater than 800 mEq/kg may provide a product having a stable shelf-life of at least twelve months.

In examples, the ozone gas is provided to the sea buckthorn oil to provide ozonated sea buckthorn oil having a refractive index equal to or greater than 1.460 at 20° C. The refractive index of the oil is determined, for example, with a refractometer.

In examples, the ozone gas is provided to the sea buckthorn oil to provide ozonated sea buckthorn oil having a volumetric density of greater than 0.91 gcm⁻³ at 20° C. and 101.3 kPa, for example a volumetric density equal to or greater than 0.92 gcm⁻³ at 20° C. and 101.3 kPa. In examples, the ozonated sea buckthorn oil has a volumetric density of from 0.92 to 0.94 gcm⁻³ at 20° C. and 101.3 kPa.

In examples, the ozone gas is provided to the sea buckthorn oil to provide ozonated sea buckthorn oil having an acid value of greater than 1 mg KOH/g. For example, the ozonated sea buckthorn oil has an acid value greater than or equal to 4.0 mg KOH/g, such as from 4.0 to 9.0 mg KOH/g.

The viscosity of the ozonated sea buckthorn oil, and whether it is provided as a stable gel, may be dependent on the degree of ozonation of the oil. In examples, the providing the ozonated sea buckthorn oil comprises providing the ozonated sea buckthorn oil in gel form.

The method comprises combining water and salt with the ozonated sea buckthorn oil. In examples, the water is deionised water. In examples, the water is purified water. In preferred examples, the water is deionised, purified water. In examples, the salt is sodium chloride.

In examples, the water and the salt are combined before being combined with the ozonated sea buckthorn oil. For example, the providing the water and the providing the salt comprise providing a salt-water solution. In preferred examples, the salt is sodium chloride, and the providing the water and the providing the salt comprises providing saline by combining the water and the sodium chloride. In particularly preferred examples, deionised, purified water is combined with sodium chloride to provide purified saline.

In preferred examples, the providing the water and the providing the salt comprises combining sodium chloride and deionised, purified water to provide purified saline. In the context of the present disclosure, where deionised, purified water and sodium chloride are used in the provision of ozonated purified saline, the ozonated purified saline is considered to comprise ozonated, deionised, purified water and sodium chloride.

In examples, the providing the water comprises providing ozonated water. For example, the water is ozonated, deionised, purified water. In examples, providing ozonated, deionised, purified water comprises supplying ozone gas to deionised, purified water (also referred to as ozonation of the water).

The ozonation of the water may be performed before or after the water is combined with the salt. Accordingly, in examples, the providing the water and the providing the salt comprises providing ozonated saline. For example, the providing the ozonated saline comprises providing ozone gas to saline. In preferred examples, the providing the water and the providing the salt comprises providing ozonated purified saline, for example by providing ozone gas to purified saline.

In examples, the providing ozonated saline comprises providing saline in a second vessel and providing ozone gas to the saline in the second vessel.

In examples, the ozone gas is provided to the saline in the second vessel until the concentration of ozone in the second vessel is at least 15 g/m³, such as at least 25 g/m³. Advantageously, the inventor has identified that supplying ozone gas to at least this concentration provides ozonated saline having characteristics suitable for use in a topical formulation base.

In examples, the pressure within the second vessel is atmospheric pressure for at least 50% of the duration of providing the ozone gas to the saline, such as at least 80% or 90% of the duration of providing the ozone gas to the saline.

In examples, the ozone gas is provided to the saline for a minimum duration. For example, the duration of providing the ozone gas is at least 5 minutes, preferably at least 10 minutes. Typically, the duration of providing the ozone gas referred to above is continuous, e.g. providing the ozone gas to the saline continuously for at least 5 minutes, preferably at least 10 minutes.

In examples, the ozone gas is provided to the saline by bubbling the ozone gas through the saline. For example, saline is provided in the second vessel, and ozone is bubbled through the saline in the second vessel. In examples, the ozone is continuously bubbled through the saline for the duration of the providing the ozone.

In examples, the ozone gas is provided with an ozone generator. For example, the second vessel is in (optionally selective) fluid communication with the ozone generator, ozone is generated by the ozone generator, and the generated ozone is supplied to the saline. In some examples, the ozone generator generates ozone from atmospheric air, and provides a fluid comprising ozone to the second vessel. In these examples, the fluid which is provided from the ozone generator to the second vessel typically comprises appreciable amounts of components other than ozone, such as nitrogen, which in the context of the present disclosure may be considered an impurity. In other, preferred, examples, the ozone generator generates ozone from an oxygen source. For example, the ozone generator is in (optionally selective) fluid communication with an oxygen source (such as an oxygen concentrator or an oxygen bottle), generates ozone from essentially pure oxygen received from the oxygen source, and provides a fluid comprising, or consisting essentially of, ozone to the second vessel. Advantageously, providing ozone to the saline with an oxygen source and ozone generator may provide an ozonated saline having a lower proportion of impurities.

The method comprises combining the water, salt, and ozonated sea buckthorn oil. For example, the water, salt, and ozonated sea buckthorn oil are combined in a third vessel. In examples, the third vessel is separate from the first vessel and the second vessel. In other examples, the third vessel is the first vessel, or the third vessel is the second vessel.

In examples, the combining comprises providing ozone gas to a mixture of the water, salt, and ozonated sea buckthorn oil. In examples, the providing the ozone gas comprises bubbling the ozone gas through the mixture in the third vessel. In examples, the ozone is continuously bubbled through mixture for the duration of the providing the ozone. In examples, the ozone is continuously bubbled through the mixture for a duration of from 30 minutes to 3 hours.

Surprisingly, the inventor has found that bubbling the ozone gas through the mixture provides a stable, homogenous blend of saline and ozonated sea buckthorn oil, thereby obviating the need for shaking/constant stirring, emulsifying agents, surfactants, or liposomal agents.

In examples, the ozone gas is provided with an ozone generator. For example, the third vessel is in (optionally selective) fluid communication with the ozone generator, ozone is generated by the ozone generator, and the generated ozone is supplied to the mixture of water, salt, and ozonated sea buckthorn oil. In some examples, the ozone generator generates ozone from atmospheric air, and provides a fluid comprising ozone to the third vessel. In these examples, the fluid which is provided from the ozone generator to the third vessel typically comprises appreciable amounts of components other than ozone, such as nitrogen, which in the context of the present disclosure may be considered an impurity. In other, preferred, examples, the ozone generator generates ozone from an oxygen source. For example, the ozone generator is in (optionally selective) fluid communication with an oxygen source (such as an oxygen concentrator or an oxygen bottle), generates ozone from essentially pure oxygen received from the oxygen source, and provides a fluid comprising, or consisting essentially of, ozone to the third vessel. Advantageously, providing ozone to the mixture with an oxygen source and ozone generator may provide a mixture having a lower proportion of impurities.

In examples, the combining comprises providing a composition comprising a dispersed phase and a continuous phase. For example, the composition is a dispersion, referring to a heterogenous system consisting of a prevalent liquid phase (dispersing phase or continuous phase) in which a liquid phase that is immiscible with the dispersing phase is dispersed (dispersed phase). For example, the composition comprises an aqueous continuous phase which comprises water, and an organic dispersed phase. Typically, the ozonated sea buckthorn oil is the dispersed phase, and the saline is the continuous phase. In examples, the composition is an oil-in-water emulsion.

In examples, the combining comprises dispersing the ozonated sea buckthorn oil as microdroplets through the water, e.g. droplets having a mean diameter of from 1 micrometre (μm) to 100 μm. In particular examples, the combining comprises bubbling ozone through the mixture of water, salt, and ozonated sea buckthorn oil and dispersing the ozonated sea buckthorn oil as microdroplets through the water. Advantageously, the inventor has identified that providing the ozonated sea buckthorn oil as microdroplets in the mixture may provide a product having improved stability.

In examples, the combining further comprises degassing the mixture of water, salt, and ozonated sea buckthorn oil. For example, the combining comprises providing ozone gas to the mixture, and subsequently degassing the mixture.

Advantageously, the inventor has identified that the degassing the mixture of water, salt, and ozonated sea buckthorn oil may reduce or avoid separation of the aqueous and organic components of the composition, for example reduces or avoids the formation of a film of ozonated sea buckthorn oil in the mixture.

As used herein, “degassing” refers to the removal of at least some dissolved or entrained gas from a liquid. In examples, degassing the mixture comprises subjecting the mixture to a reduced pressure environment, thereby liberating the dissolved or entrained gas from the liquid. For example, the mixture is provided in the third vessel, and the pressure within the vessel is reduced by, for example, applying a vacuum to the third vessel. Through application of a vacuum to the third vessel, gas which was dissolved or entrained in the liquid before degassing is liberated from the liquid and removed from the third vessel. In examples, the third vessel is a vacuum chamber (also referred to as a vacuum degasser).

Accordingly, in some examples, for at least 50% of a duration of degassing the mixture, a pressure within the third vessel is less than atmospheric pressure, such as at least 80% or 90% of the duration of degassing the mixture. In examples, the degassing comprises reducing the pressure within the third vessel to less than atmospheric pressure for a period of at least 5 minutes per 500 mL of liquid in the third vessel.

In examples, the degassing is performed as a batch degassing operation, for example, using a vacuum chamber and pump. In other examples, the degassing is performed as a continuous degassing operation, for example using a membrane degasser.

In some examples, a vacuum mixer or vacuum blender may be used in performing the degassing.

In other examples, degassing the mixture comprises applying ultrasonic radiation to the mixture (sometimes referred to as ultrasonic degassing), and/or sparging the mixture with another gas, such as nitrogen, argon, helium, or other inert gases.

In examples, the degassing comprises stirring and/or agitating the mixture while removing at least some dissolved or entrained gas from the mixture.

In examples, the combining the water, salt, and ozonated sea buckthorn oil comprises bubbling ozone gas through a mixture of water, salt, and ozonated sea buckthorn oil, and subsequently degassing the mixture, wherein the degassing comprises removing at least some of the ozone gas dissolved or entrained in the mixture from the bubbling of ozone gas through the mixture, such as at least 50% by weight, at least 90% by weight, or substantially all, of the ozone gas that is dissolved or entrained in the mixture. Further, in examples, the degassing comprises removing at least some nitrogen and oxygen from the mixture.

Surprisingly, as noted above, the inventor has identified that the methods disclosed herein may obviate the need for including certain preservatives, fragrances, alcohols, emulsifiers, and/or surfactants in the provision of a topical formulation base. Advantageously, reducing the content of such components in the topical formulation base compared with those known in the art, or omitting such components entirely from the topical formulation base, may reduce the risk of a human experiencing a sensitising reaction deriving from topical application of a topical formulation comprising the topical formulation base.

In examples, the method comprises combining no more than 5 wt % (taken together, based on the total weight of the formulation base) a preservative, a fragrance, an alcohol, an emulsifier, and/or a surfactant with the water, salt, and ozonated sea buckthorn oil. In examples, the method does not comprise combining any of a preservative, a fragrance, or an alcohol with the water, salt, and ozonated sea buckthorn oil.

In examples, the method does not comprise combining a preservative, a fragrance, an alcohol, an emulsifier, and/or a surfactant with the water, salt, and ozonated sea buckthorn oil. In examples, the method does not comprise combining any of a preservative, a fragrance, or an alcohol with the water, salt, and ozonated sea buckthorn oil.

In examples, the preservative is selected from the list consisting of: benzalkonium chloride, 2-Bromo-2-nitropropane-1,3-diol, chlorhexidine digluconate, chlorphenesin, dehydroacetic acid, diazolidinyl urea, dmdm-hydantoin [1,3-bis(hydroxy-methyl)-5,5-dimethylimidazolidine-2,4-dione] ethylhexylglycerin, formaldehyde-releasing compounds (e.g, dmdm-hydantoin, quaternium-15, imidazolidinyl urea, diazolidinyl urea, and 2-bromo-2-nitropropane-1,3-diol), imidazolidinyl urea, methyldibromo glutaronitrile, methylisothiazolinone, phenoxyethanol, quaternium-15, sodium benzoate, sodium hydroxy-methylglycinate, sorbic acid, thimerosal, triclosan, and combinations thereof. In examples, the method does not include combining any of the preservatives recited hereinabove with the water, salt, and ozonated sea buckthorn oil.

In examples, the fragrance is selected from the list consisting of: parfum, linalool, limonene, citronellol, geraniol, eugenol, rose extract (Rosa damascena), lavender oil (Lavandula angustifolia), ylang-ylang oil (Canaga odorata), and combinations thereof. In examples, the method does not include combining any of the fragrances recited hereinabove with the water, salt, and ozonated sea buckthorn oil.

In examples, the alcohol is selected from the list consisting of: ethanol, isopropyl alcohol, and combinations thereof. In examples, the method does not include combining any of the alcohols recited hereinabove with the water, salt, and ozonated sea buckthorn oil.

In examples, the emulsifier is selected from the list consisting of: lecithin, phospholipid, and caseinate, and combinations thereof. In examples, the method does not include combining any of the emulsifiers recited hereinabove with the water, salt, and ozonated sea buckthorn oil.

In examples, the surfactant is selected from the list consisting of: polyoxyethylene (80) sorbitan monooleate, sodium lauryl sulfate, ammonium lauryl sulfate, or their ethoxylated companions, sodium laureth sulfate, and combinations thereof. In examples, the method does not include combining any of the surfactants recited hereinabove with the water, salt, and ozonated sea buckthorn oil.

In examples, the combining the water, salt, and ozonated sea buckthorn oil provides a composition comprising from 0.1% to 10% w/w ozonated sea buckthorn oil, such as 0.5 to 5% w/w ozonated sea buckthorn oil. The inventor has identified that, advantageously, a composition having such an ozonated sea buckthorn content is particularly suitable for use as a topical formulation base.

In examples, the combining the water, salt, and ozonated sea buckthorn oil provides a composition comprising water in an amount of from 80 to 99.8% w/w, preferably 90 to 99.8% w/w, more preferably 95 to 99.8% w/w, even more preferably 98 to 99.8% w/w.

In examples, the combining the water, salt, and ozonated sea buckthorn oil provides a composition comprising salt in an amount of from 0.1% to 5% w/w, preferably 0.5 to 2% w/w, more preferably 0.7 to 1.2% w/w.

For example, the water, salt, and ozonated sea buckthorn oil are combined such that the composition comprises water in an amount of from 98 to 99% w/w, ozonated sea buckthorn oil in an amount of from 0.2 to 0.8% w/w, and salt in an amount of from 0.7 to 1.2% w/w. In particular examples, the composition comprises approximately 98.6% w/w de-ionised ozonated water, 0.5% w/w ozonated sea buckthorn oil, and 0.9% w/w sodium chloride.

In examples, the providing the water, salt, and ozonated sea buckthorn oil comprises providing them in respective amounts that correspond to the amounts present in the composition described hereinabove.

In particular examples, the method comprises: providing ozone gas to sea buckthorn oil in a first vessel for a duration of at least 12 hours at a flow rate of at least 100 L/h to provide ozonated sea buckthorn oil having a peroxide value equal to or greater than 800 mEq/kg, wherein the providing the ozone gas comprises bubbling the ozone gas through the sea buckthorn oil; combining deionised, purified water and sodium chloride to provide purified saline; providing ozone gas to the purified saline in a second vessel from an oxygen source generator for a duration of at least 5 minutes to provide ozonated purified saline, wherein the providing the ozone gas comprises bubbling the ozone gas through the purified saline; combining the ozonated purified saline with the ozonated sea buckthorn oil in a third vessel, to provide a composition comprising water in an amount of from 80 to 99.8% w/w of the composition, sodium chloride in an amount of 0.05 to 5% w/w of the composition, and ozonated sea buckthorn oil in an amount of from 0.1 to 5% w/w of the composition, wherein the combining comprises: providing ozone gas to a mixture of the ozonated purified saline and the ozonated sea buckthorn oil in the third vessel; wherein the providing ozone gas to the mixture comprises bubbling the ozone gas through the mixture; and degassing the mixture in the third vessel.

In examples, the combining does not comprise combining any components other than the water, salt, and ozonated sea buckthorn oil. For example, the composition consists essentially of the water, salt, and ozonated sea buckthorn oil. Such a composition may be particularly suitable as a topical formulation base in the manufacture of certain topical formulations.

In other examples, the combining comprises combining one or more further topical formulation base components with the water, salt, and ozonated sea buckthorn oil to provide a composition comprising no more than 10% w/w of the one or more further topical base components, such as more than 5% w/w, 1% w/w, or 0.1% w/w of the one or more further topical formulation base components.

In examples, the composition comprises no more than 5% w/w, such as no more than 1% w/w, or no more than 0.1% w/w components other than the water, salt, and ozonated sea buckthorn oil.

In examples, the one or more further topical formulation base components comprises a stabiliser and/or an emulsifier, such that the method comprises combining the stabiliser and/or emulsifier with the water, salt, and ozonated sea buckthorn oil, thereby providing a composition comprising water, salt, ozonated sea buckthorn oil, and stabiliser and/or emulsifier.

In examples, the composition comprises the stabiliser in an amount of from 0.1% to 5% w/w. For example, the composition comprises hydroxyethyl acrylate/sodium acryloyldimethyl taurate copolymer (such as Sepinov™ EMT 10, available from SEPPIC), in an amount of from 0.1% to 5% w/w. In examples, the composition comprises the emulsifier in an amount of from 0.1% to 5% w/w. For example, the composition comprises a polyoxyethylene, polyoxypropylene block polymer (such as Poloxamer 184 (available from Merck KGaA) in an amount of from 0.1% to 5% w/w. In examples, the composition comprises water, salt, ozonated sea buckthorn oil, stabiliser, and emulsifier.

In examples, the composition comprises stabiliser and/or emulsifier (taken together, if both present) in an amount of from 0.1% to 10% w/w, such as 1% to 8% w/w.

In particular examples, the composition comprises approximately 92.6% w/w de-ionised ozonated water, 3.0% w/w Sepinov™ EMT 10, 3.0% w/w Poloxamer 184, 0.5% w/w ozonated sea buckthorn oil, and 0.9% w/w sodium chloride. The composition of these examples may be particularly suited for use as the topical formulation base for an eye cleanser topical formulation.

In examples of the method, after the combining, one or more further processes are performed to provide the topical formulation base. In examples, the one or more further processes comprises irradiating the composition, agitating the composition, and/or providing the composition in a container.

According to a second aspect of the present disclosure, there is provided a method for providing a topical formulation, the method comprising performing the method of the first aspect to provide a topical formulation base; and combining the topical formulation base with one or more topical formulation components.

In examples, the topical formulation comprises the topical formulation base in an amount of at least 70% w/w of the topical formulation.

In examples, the one or more topical formulation components comprises: an active ingredient; an oil; a surfactant; or a combination thereof.

In examples, the method further comprises, after the combining the topical formulation base with the one or more topical formulation components, performing one or more further processes to provide the topical formulation. In examples, the one or more further processes comprises irradiating the composition, agitating the composition, and/or providing the composition in a container.

According to a third aspect of the present disclosure, there is provided a composition obtainable from the method of the first or second aspects.

According to a fourth aspect of the present disclosure, there is provided a composition comprising: water in an amount of 80 to 99.8% w/w; salt in an amount of 0.1 to 5% w/w; and ozonated sea buckthorn oil in an amount of 0.1 to 10% w/w. In examples, the composition is obtained or obtainable from the method of the first or second aspect. In examples, the composition according to the fourth aspect is the composition according to the third aspect.

In examples, the composition comprises water, salt, and ozonated sea buckthorn oil in the proportions described hereinabove in relation to the first aspect. Similarly, in examples, the components of the composition have one or more of any of the characteristics described hereinabove in relation to the first aspect.

For example, the ozonated sea buckthorn oil of the composition has a peroxide value equal to or greater than 300 mEq/kg, preferably equal to or greater than 600 mEq/kg, more preferably greater than or equal to 800 mEq/kg, still more preferably greater than or equal to 1,500 mEq/kg. In examples, the ozonated sea buckthorn oil has a peroxide value equal to or less than 2,200 mEq/kg.

In examples, the ozonated sea buckthorn oil of the composition has a refractive index equal to or greater than 1.460 at 20° C.

In examples, the ozonated sea buckthorn oil of the composition has a volumetric density of greater than 0.91 gcm⁻³ at 20° C. and 101.3 kPa, for example a volumetric density equal to or greater than 0.92 gcm⁻³ at 20° C. In examples, the ozonated sea buckthorn oil has a volumetric density of from 0.92 to 0.94 gcm⁻³ at 20° C. and 101.3 kPa.

In examples, the ozonated sea buckthorn oil of the composition has an acid value of greater than 1 mg KOH/g. For example, the ozonated sea buckthorn oil has an acid value greater than or equal to 4.0 mg KOH/g, such as from 4.0 to 9.0 mg KOH/g.

In particular examples, the composition comprises ozonated purified water in an amount of 80 to 99.8% w/w of the composition, sodium chloride in an amount of 0.05 to 5% w/w of the composition, and ozonated sea buckthorn oil in an amount of 0.1 to 5% w/w of the composition, wherein the ozonated sea buckthorn oil has a peroxide value equal to or greater than 800 mEq/kg.

In examples, the composition comprises, taken together, no more than 5% w/w a preservative, a fragrance, an alcohol, an emulsifier, and/or a surfactant as described hereinabove in relation to the first aspect.

In examples, the composition consists essentially of the ozonated deionised, purified water, sodium chloride, and ozonated sea buckthorn oil.

In examples, the composition is a liquid. For example, comprises ozonated sea buckthorn oil dispersed through saline. In examples, the composition is an oil-in-water emulsion.

In examples, the composition is a topical formulation base. In examples, the composition is a topical formulation. In some examples, a topical formulation base is a topical formulation. For example, the topical formulation base is suitable for direct use as a topical formulation without further modification of the composition.

According to a fifth aspect of the present disclosure, there is provided a topical formulation base comprising the composition according to the fourth aspect.

In examples, the topical formulation base comprises, taken together, no more than 5% w/w a preservative, a fragrance, an alcohol, an emulsifier, and/or a surfactant as described hereinabove in relation to the first aspect. In examples, the topical formulation base is essentially free of preservative, fragrance, alcohol, emulsifier, and surfactant as described hereinabove in relation to the first aspect

In examples, the topical formulation base consists essentially of the ozonated deionised, purified water, sodium chloride, and ozonated sea buckthorn oil as described hereinabove.

According to a sixth aspect of the present disclosure, there is provided a topical formulation comprising the composition according to the fourth aspect, and one or more topical formulation components in an amount of no more than 30% w/w. In examples, the one or more topical formulation components are as described hereinabove in relation to the second aspect.

In examples, the topical formulation comprises, taken together, no more than 5% w/w a preservative, a fragrance, an alcohol, an emulsifier, and/or a surfactant as described hereinabove in relation to the first aspect. In examples, the topical formulation is essentially free of preservative, fragrance, and alcohol as described hereinabove in relation to the first aspect.

In examples, the one or more topical formulation components does not include water, salt, or ozonated sea buckthorn oil. For example, the water, salt, and ozonated sea buckthorn oil present in the topical formulation are provided only from the topical formulation base which is present in the topical formulation.

In examples, the topical formulation base of the topical formulation does not comprise any components corresponding to the one or more topical formulation components. For example, the one or more topical formulation components present in the topical formulation do not derive from the topical formulation base which is present in the topical formulation.

In examples, the topical formulation is an eye lid cleanser, an eye lash cleanser, an eye serum, an eye cream, an eyelash serum, an eye mask, a contact lens solution, a contact lens cleaner, an eye spray, and/or an eye drop.

In examples, the topical formulation is a liquid, gel, foam, cream.

According to a seventh aspect of the present disclosure, there is provided a kit comprising an applicator and the topical formulation of the sixth aspect. In examples, the applicator is configured for topical application of the formulation. In particular, the applicator is configured for directed topical application according to the intended use of the topical formulation.

In examples, the applicator is configured as a bottle suitable for dispensing the topical formulation to a pad, wipe, or the like, to subsequently be used for topical application of the formulation by the user. These examples may be particularly suitable where the topical formulation is a cleanser.

In examples, the applicator comprises a dispenser for dispensing the topical formulation to a user. For example, the dispenser includes a pump mechanism. These examples may be particularly suitable where the topical formulation is a cream. In other examples, the dispenser includes a pipette or dropper. These examples may be particularly suitable where the topical formulation is a serum, contact lens solution, contact lens cleaner, or eye drop. In further examples, the dispenser includes an atomiser. This example may be particularly suitable where the topical formulation is an eye spray.

In examples, the applicator is a porous membrane which is impregnated with the topical formulation, such as a “wet wipe”, or a topical occlusive patch.

According to an eighth aspect of the present disclosure, there is provided a non-therapeutic method of providing an anti-microbial and/or anti-fungal effect to skin, the method comprising topical application of the topical formulation of the sixth aspect to a human. In examples, the topical application comprises applying the formulation to the face of the human, for example periorbital region of the face.

Without wishing to be bound by theory, it is believed that the formulation of the present disclosure is particularly suitable for applying to the area of the face surrounding and including the eyes, because the eye lid margin is vulnerable to bacterial infection and inflammation (causing dry eyes). The anti-microbial and/or anti-fungal properties of the composition may reduce and/or avoid such bacterial infection.

Also provided herein is use of the composition, topical formulation base, or topical formulation as a cosmetic product. For example, there is provided the use of the topical formulation in cosmetic, or non-therapeutic methods. Exemplary non-therapeutic methods include cleansing of the skin, hair follicles, and reduction or avoidance of skin aging. In examples, the non-therapeutic method comprises topical application of the formulation, e.g. application of the formulation to an external surface of the skin. In particular examples, the non-therapeutic method comprises topical application of the formulation to the skin of a user's face, in particular the periorbital region of the face.

According to a ninth aspect of the present disclosure, there is provided the composition, topical formulation base, or topical formulation described hereinabove for use as a medicament. For example, provided herein is the composition, topical formulation base, or topical formulation for use in a therapeutic method.

In examples, the composition, topical formulation base, or topical formulation is for use in a therapeutic method of preventing rosacea, eczema, erythema, or cutaneous sensory syndrome (also referred to as sensitive skin, a skin condition wherein the skin is hypersensitive to stimuli, presenting with itching, irritant, erythema, and dryness).

In examples, the composition, topical formulation base, or topical formulation is for use in a therapeutic method of reducing rosacea, eczema, erythema, or cutaneous sensory syndrome (also referred to as sensitive skin, a skin condition wherein the skin is hypersensitive to stimuli, presenting with itching, irritant, erythema, and dryness). For example, the reducing comprises reduction of the adverse symptoms experienced by a subject.

In examples, the therapeutic method comprises topical application of the formulation, e.g. application of the formulation to an external surface of the skin. In particular examples, the therapeutic method comprises topical application of the formulation to the skin of a user's face, in particular the periorbital region of the face.

In examples, the therapeutic method or non-therapeutic method described hereinabove comprises administering the composition to the skin of a patient in place of a second composition comprising non-ozonated sea buckthorn oil. For example, the composition is administered instead of the second composition. Put another way, the composition is administered as a substitute, or replacement, of the second composition in a method of topical application of the second composition.

In examples, the therapeutic method or non-therapeutic method described hereinabove comprises administering the composition in place of a second composition, the composition differing from the second composition only in that it comprises ozonated sea buckthorn oil. In examples, the composition differs from the second composition only in that it comprises ozonated sea buckthorn oil instead of non-ozonated sea buckthorn oil. For example, other than the ozonated or non-ozonated sea buckthorn oil, the composition and the second composition consist of the same constituents in the same proportions.

In examples, the therapeutic method or non-therapeutic method described hereinabove comprises administering the composition in place of a second composition comprising preservatives, fragrances, alcohols, emulsifiers, and surfactants in an amount of at least 50% w/w of the composition, taken together, or at least 30% w/w, or at least 10% w/w.

In examples, the therapeutic method or non-therapeutic method described hereinabove comprises administering the composition in place of a second composition comprising surfactant and/or emulsifier in an amount of at least 20% w/w of the composition, taken together, or at least 10% w/w, or at least 5% w/w.

In examples, the therapeutic method or non-therapeutic method described hereinabove comprises administering the composition in place of a second composition comprising preservatives, fragrances, and/or alcohols in an amount of at least 50% w/w of the composition, taken together, or at least 30% w/w, or at least 10% w/w. In examples, the preservatives, fragrances, and/or alcohols are selected from the preservatives, fragrances, alcohols described hereinabove.

Features described herein in relation to one aspect of the present disclosure are explicitly disclosed in combination with the other aspects, to the extent that they are compatible

Further features and advantages of the invention will become apparent from the following description of preferred embodiments of the invention, given by way of example only, which is made with reference to the accompanying drawings.

### BRIEF DESCRIPTION OF THE DRAWINGS

Embodiments of the invention will now be described, by way of example only, with reference to the accompanying drawings, in which:

FIG. 1 shows a schematic apparatus for performing a method of providing a topical formulation base according to examples;

FIG. 2 shows a schematic flow chart of a method of providing a topical formulation base according to examples;

FIG. 3 shows a schematic flow chart of a method of providing a topical formulation according to examples;

FIG. 4 shows a schematic kit according to examples;

FIGS. 5 to 8 are charts showing data obtained from the qualitative survey of Example 5; and

FIG. 9 is a chart showing data obtained from the topical preservative efficacy test (PET) of Example 6.

### DETAILED DESCRIPTION OF CERTAIN INVENTIVE EMBODIMENTS

Percentages of components in compositions as provided herein refer to the percentage of that component of the composition by weight (w/w), unless indicated otherwise.

Typically, sea buckthorn oil is obtained from the berries of the sea buckthorn plant (Hippophae Rhamnoides). In examples, at least part, or all, of the sea buckthorn oil is obtained from the seeds of the sea buckthorn berry. In examples, at least part, or all, of the sea buckthorn oil is obtained from the pulp and/or peel of the sea buckthorn berry. In examples, the sea buckthorn oil is obtained from cold-pressing of components of sea buckthorn berries, such as cold-pressing of sea buckthorn seeds.

Non-ozonated sea buckthorn oil obtained from the seeds of the berry may comprise fatty acids in the following ranges (percentages given as weight percent of fatty acids present in the oil):

TABLE 1

| Common name | Lipid name | Wt % |
|---|---|---|
| Palmitic acid | 16:0 | 6-9 |
| Steric acid | 18:0 | 1-5 |
| Oleic acid | 18:1 (n-9) | 15-19 |
| Vaccenic acid | 18:1 (n-7) | 2-4 |
| Linoleic acid | 18:2 (n-6) | 35-43 |
| α-Linoleic acid | 18:3 (n-3) | 26-34 |

Non-ozonated sea buckthorn oil obtained from the pulp and/or peel of the berry may comprise fatty acids in the following ranges (percentages given as weight percent of fatty acids present in the oil):

TABLE 2

| Common name | Lipid name | Wt % |
|---|---|---|
| Palmitic acid | 16:0 | 23-32 |
| Palmitoleic acid | 16:1 (n-7) | 28-36 |
| Steric acid | 18:0 | 0.5-1.5 |
| Oleic acid | 18:1 (n-9) | 14-21 |
| Vaccenic acid | 18:1 (n-7) | 7-11 |
| Linoleic acid | 18:2 (n-6) | 5-13 |
| α-Linoleic acid | 18:3 (n-3) | 1-5 |

Non-ozonated sea buckthorn oil obtained from the whole berry (including seed and pulp/peel) may comprise fatty acids in the following proportions (percentages given as weight percent of fatty acids present in the oil):

TABLE 3

| Common name | Lipid name | Wt % |
|---|---|---|
| Palmitic acid | 16:0 | 19-28 |
| Palmitoleic acid | 16:1 (n-7) | 22-30 |
| Steric acid | 18:0 | 1-2 |
| Oleic acid | 18:1 (n-9) | 15-20 |
| Vaccenic acid | 18:1 (n-7) | 5-9 |
| Linoleic acid | 18:2 (n-6) | 11-19 |
| α-Linoleic acid | 18:3 (n-3) | 5-10 |

Iodine Value (IV) is a measure of the total number of double bond present in a sample. It represents the quantity of iodine (in grammes) that will react with the double bonds in 100 g of sample. Iodine values provided herein were experimentally obtained through titration against potassium thiosulfate, and calculated according to the following equation:

$$1. 269 \times ( {n}_{1} - {n}_{2} ) / m$$

where n₁ is the volume in mL of potassium thiosulfate solution used to carry out a blank test, n₂ is the volume in mL of potassium thiosulfate solution used for the titration and m is the quantity in grammes of the oil titrated against.

Acid Value (AV) is a number that expresses in milligrammes (mg) the quantity of potassium hydroxide required to neutralize the free acids present in 1 g of an oil sample. Avid values were experimentally obtained through titration against potassium hydroxide, and calculated according to the following equation:

$$5.61 \times n / m$$

where n is the volume in mL of the titrant and m the quantity in grammes of the oil titrated against.

Peroxide Value (PV) represents the quantity of peroxide expressing in milliequivalents of active oxygen contained in 1 kg of an oil sample.

Peroxide values provided herein were experimentally obtained through titration against potassium thiosulfate, and calculated according to the following equation:

$$1 , 000 \times ( {V}_{1} - {V}_{0} ) \times c / m$$

where V₁ is the volume in mL of potassium thiosulfate solution used for the titration, V₀ is the volume in mL of potassium thiosulfate solution used to carry out a blank test, c is the potassium thiosulfate concentration and m is the quantity in grammes of the oil sample titrated against.

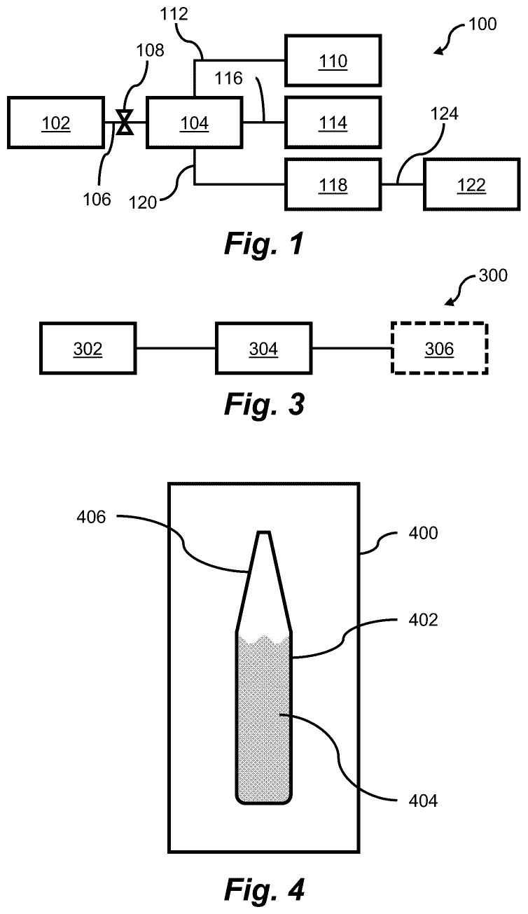

FIG. 1 shows a schematic apparatus 100 for performing a method of providing a topical formulation base according to examples. In this example, the apparatus comprises an oxygen source 102. Typically, the oxygen source 102 is essentially pure oxygen contained in an oxygen container. Alternatively, the oxygen source 102 is an oxygen concentrator, configured to concentrate oxygen from ambient air by selectively removing nitrogen. In this example, the oxygen source 102 is an oxygen bottle containing medical-grade oxygen.

The oxygen source 102 is selectively fluidically connected to an ozone generator 104. For example, the oxygen source 102 is connected to the ozone generator 104 via a conduit 106, and a valve 108 arranged along the conduit 106 between the oxygen source 102 and the ozone generator 104. Means of controlling the fluidic flow between the oxygen source 102 and the ozone generator 104 other than the valve 108 are contemplated. The oxygen source 102, ozone generator 104, and conduit 106 are configured such that oxygen from the oxygen source 102 flows to the ozone generator 104 in use. The ozone generator 104 is configured to generate ozone from the oxygen received from the oxygen source.

In this example, the ozone generator 104 is a corona discharge ozone generator, such as the 1KNT ozone generator available from Oxidation Technologies, LLC., Iowa, USA.

The ozone generator 104 is selectively fluidically connected to a first vessel 110 via conduit 112, such that ozone generated with the ozone generator 104 can flow from the ozone generator 104 through the conduit 112 to the first vessel 110. Optionally, a valve is arranged along the conduit 112 between the first vessel 110 and the ozone generator 104 (not shown). Typically, the first vessel 110 is a glass bubble column with a rubber seal through which the conduit 112 extends, such that the first vessel 110 can be made gas-tight to the surrounding atmosphere while receiving ozone from the ozone generator 104. In use, the first vessel 110 is charged with sea buckthorn oil, the first vessel 110 sealed to be gas-tight to the surrounding atmosphere, and ozone provided to the sea buckthorn oil from the ozone generator 104 to provide ozonated sea buckthorn oil. The conduit 112 is arranged at the first vessel 110 such that, in use, ozone flowing from the conduit bubbles through the sea buckthorn oil.

The ozone generator 104 is selectively fluidically connected to a second vessel 114 via conduit 116, such that ozone generated with the ozone generator 104 can flow from the ozone generator 104 through the conduit 116 to the second vessel 114. Optionally, a valve is arranged along the conduit 116 between the second vessel 114 and the ozone generator 104 (not shown). Typically, the second vessel 110 is a glass bubble column with a rubber seal through which the conduit 116 extends, such that the second vessel 114 can be made gas-tight to the surrounding atmosphere while receiving ozone from the ozone generator 104. In use, the second vessel 114 is charged with saline, the second vessel 114 sealed to be gas-tight to the surrounding atmosphere, and ozone provided to the saline from the ozone generator 104 to provide ozonated saline. The conduit 116 is arranged at the second vessel 114 such that, in use, ozone flowing from the conduit bubbles through the saline.

In the example shown in FIG. 1, the apparatus 100 may be configured to prepare ozonated sea buckthorn oil and ozonated saline concurrently. Alternatively, the apparatus 100 may be configured to prepare ozonated sea buckthorn oil and ozonated saline sequentially, for example first provide ozone to the sea buckthorn oil in the first vessel, then provide ozone to the saline in the second vessel 114. This may be achieved, for example, when the conduit 112 connected to the first vessel 110 is the same as the conduit 116 connected to the second vessel 114; in use the conduit 112, 116 is disconnected from the first vessel 110 after providing ozone to the sea buckthorn oil and then connected to the second vessel 114 such that ozone can be provided from the ozone generator 104 to the saline.

In the example shown, the first vessel 110 is separate from the second vessel 114. In some examples (not shown) the first vessel 110 and the second vessel 114 are the same vessel. For example, after ozonation of sea buckthorn oil in the vessel 110, 114, the ozonated sea buckthorn oil is removed from the vessel 110, 114 and water charged to the vessel 110, 114 to then receive ozone from the ozone generator 104.

The ozone generator 104 is selectively fluidically connected to a third vessel 118 via conduit 120, such that ozone generated with the ozone generator 104 can flow from the ozone generator 104 through the conduit 120 to the third vessel 118. Optionally, a valve is arranged along the conduit 120 between the third vessel 118 and the ozone generator 104 (not shown). Typically, the third vessel 118 is a glass bubble column with a rubber seal through which the conduit 120 extends, such that the third vessel 118 can be made gas-tight to the surrounding atmosphere while receiving ozone from the ozone generator 104. In use, the third vessel 114 is charged with ozonated sea buckthorn oil (e.g. provided from the first vessel 110) and ozonated saline (e.g. provided from the second vessel 114), the third vessel 118 sealed to be gas-tight to the surrounding atmosphere, and ozone provided to the mixture of ozonated sea buckthorn oil and ozonated saline from the ozone generator 104. The conduit 118 is arranged at the third vessel 118 such that, in use, ozone flowing from the conduit bubbles through the mixture. Bubbling ozone through the mixture may provide a well-dispersed distribution of ozonated sea buckthorn oil microdroplets throughout the ozonated saline.

In examples, the conduit 120 connected to the third vessel is the conduit 116 connected to the second vessel 110 and/or the conduit 112 connected to the first vessel 110. For example, in use, the conduit 116, 120 is disconnected from the second vessel 114 after providing ozone to the saline and then connected to the third vessel 118 such that ozone can be provided from the ozone generator 104 to the mixture of ozonated sea buckthorn oil and ozonated saline in the third vessel 118.

In the example shown, the third vessel 118 is separate from the first vessel 110 and the second vessel 114. In other examples (not shown), the third vessel 118 is the first vessel 110. For example, in use, ozonated saline from the second vessel 114 is combined with the ozonated sea buckthorn oil in the first vessel 110. In further examples (not shown), the third vessel 118 is the second vessel 114. For example, in use, ozonated sea buckthorn oil from the first vessel 110 is combined with the ozonated saline in the second vessel 114.

Typically, while supplying ozone from the ozone generator 104 to the vessels 110, 114, 118, the pressure within the vessel 110, 114, 118 is greater than atmospheric pressure.

In use, the ozone generator 104 is configured to supply fluid comprising ozone to the first vessel 110, second vessel 114, and/or third vessel 118 at a flow rate of 200 L/h.

In other examples (not shown), the ozone generator 104 is configured to generate ozone from ambient air provided to the ozone generator 104 via, for example, a port in the housing of the ozone generator 104. In these examples, the fluid supplied from the ozone generator 104 to the first vessel 110, second vessel 114, and/or third vessel 118 may contain a higher proportion of impurities (e.g. non-ozone constituents) compared with the example where oxygen is provided to the ozone generator 104 from an oxygen source 102 comprising essentially pure oxygen.

The apparatus 100 further comprises a vacuum source 122 in selective fluidic communication with the third vessel 118 via conduit 124. The vacuum source 122 is configured to reduce the pressure in the third vessel 118 in use. The apparatus 100 is configured such that, in use, after ozone ceases to be supplied from the ozone generator 104 to the third vessel 118, the vacuum source 122 exerts negative pressure on the content of the third vessel 118, thereby degassing the mixture residing in the third vessel 118.

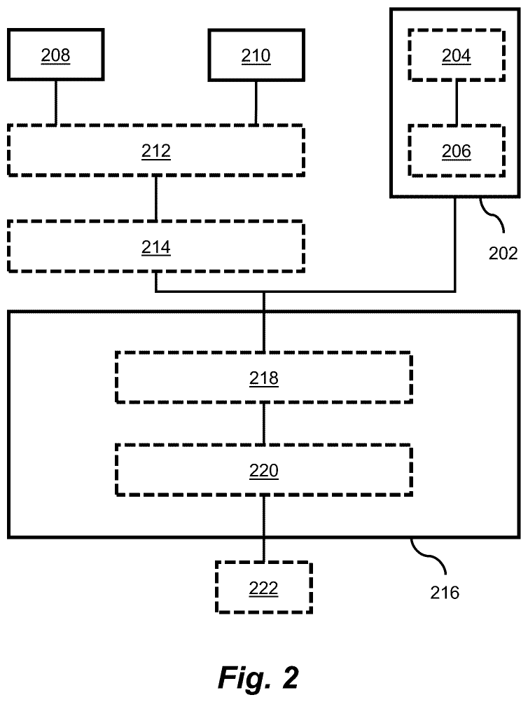

FIG. 2 shows a schematic flow chart of a method 200 of providing a topical formulation base according to examples. The method 200 depicts optional parts of the method in dashed lines. In examples, the method 200 may be performed with the apparatus 100 depicted in FIG. 1. To aid understanding, reference numerals from FIG. 1 may be used in describing the method 200.

The method 200 comprises providing 202 ozonated sea buckthorn oil. In this example, providing 202 ozonated sea buckthorn oil comprises providing 204 (non-ozonated) sea buckthorn oil in the first vessel 110, and providing 206 ozone gas to the sea buckthorn oil to afford ozonated sea buckthorn oil, e.g. with the ozone generator 104 of the apparatus 100. In this example, the providing 206 ozone to the sea buckthorn oil comprises bubbling the ozone through the sea buckthorn oil in the first vessel, and is performed for a continuous period of at least 12 hours such that the afforded ozonated sea buckthorn oil has a peroxide value greater than or equal to 800 mEq/kg.

The method 200 also comprises providing 208 water and providing 210 salt. In this example the providing 210 salt comprises providing sodium chloride. Further, in this example, the providing 208 water comprises providing deionised, purified water.

The method 200 further comprises combining 212 the water and salt to provide saline. The combining 212 is performed, for example, in the second vessel 114 of the apparatus 100. In this example, the combining 212 is performed to provide saline having a sodium chloride content of approximately 0.91% w/w.

The method further comprises providing 214 ozone gas to the (non-ozonated) saline in the second vessel 114 to afford ozonated saline, e.g. with the ozone generator 104 of the apparatus 100. In this example, the providing 214 ozone to the saline comprises bubbling the ozone through the saline for a continuous period of at least 5 minutes to afford ozonated saline.

The method further comprises combining 216 the ozonated saline and the ozonated sea buckthorn oil. In this example, the combining 216 is performed to provide a composition comprising 98.6% w/w water, 0.9% w/w sodium chloride, and 0.5% ozonated sea buckthorn oil. The combining 216 comprises providing 218 ozone gas to a mixture of the ozonated saline and the ozonated sea buckthorn oil in the third vessel 118, e.g. with the ozone generator 104 of the apparatus 100. In this example, the providing 218 ozone to the mixture comprises bubbling the ozone through the mixture for a continuous period of 0.5 to 3 hours. The combining 216 further comprises degassing 220 the mixture in the third vessel 118, e.g. with the vacuum source 122 of the apparatus 100. The degassing 220 typically comprises reducing the pressure in the third vessel 118 to a pressure less than atmospheric pressure, thereby liberating at least some dissolved or entrained gas from the mixture, and removing at least some of the liberated gas from the third vessel 118 (and thus the mixture) with the vacuum source 122. In this example, the degassing 220 is performed for a continuous period of at least 5 minutes per 500 mL of liquid in the third vessel 118.

Typically, the combining 216 provides a topical formulation base with microdroplets of ozonated sea-buckthorn oil dispersed throughout the saline.

In examples, the method 200 comprises performing 222 one or more further processes on the composition obtained from the combining 216 to provide a topical formulation base. For example, the performing 218 one or more further processes comprises irradiating the composition, agitating the composition, and/or providing the composition in a container.

FIG. 3 shows a schematic flow chart of a method 300 of providing a topical formulation according to examples. The method 300 comprises providing 302 a topical formulation base. In this example, the providing 302 the topical formulation base comprises performing method 200 depicted in FIG. 2. The method 300 further comprises combining 304 the topical formulation base with one or more topical formulation components. In examples, the combining 304 comprises combining the topical formulation base with one or more of an active ingredient, an oil, a surfactant, or a combination thereof. In examples, the method 300 further comprises performing 304 one or more further processes on the composition obtained from the combining 302 to provide a topical formulation. For example, the performing 304 one or more further processes comprises irradiating the composition, agitating the composition, and/or providing the composition in a container.

FIG. 4 shows a schematic kit 400 according to examples. The kit 400 comprises an applicator 402 and a topical formulation 404. Typically, the topical formulation 404 is the topical formulation obtained from the method 300 depicted in FIG. 3. In other examples, the topical formulation 404 is the topical formulation base obtained from the method 200 depicted in FIG. 2 which is suitable for use as both a topical formulation base and as a topical formulation as such. In this example, the applicator 402 is configured for topical application of the formulation by comprising a dropper portion 406 to direct application of the topical formulation. However, other configurations of applicator are envisaged.

### EXAMPLES

### Example 1-Preparation of Topical Formulation Base Compositions

Topical formulation base compositions were prepared according to the following protocol in a cold room (ambient temperature less than 17° C.). Ozone was generated with an ozone generator using pure oxygen (medical grade) supplied to the oxygen generator from an oxygen bottle.

### (a) Provision of Ozonated Sea Buckthorn Oil

500 mL of sea buckthorn oil was provided in a glass bubble column. The glass bubble column was fluidically connected to the ozone generator and sealed (made gas-tight to the external atmosphere) with a rubber top. Ozone was supplied to the glass beaker from the oxygen generator at a rate of 200 litres per hour (L/h) per 10 litres of oil with an ozone concentration of 25 g/m³ and bubbled through the sea buckthorn oil for a predetermined duration. The pressure in the gas-tight beaker was between 0.6 and 0.7 bar for the duration of ozone bubbling. After the predetermined duration had elapsed, ozonated sea buckthorn oil was afforded as a low viscosity oil or stable gel.

### (b) Provision of Ozonated Saline

Purified, deionised water was mixed with sodium chloride. The saline was provided to a glass cylinder and filled 75% of the internal volume. The glass cylinder was fluidically connected to the ozone generator and made gas-tight. Ozone was supplied to the glass cylinder from the oxygen generator and bubbled through the saline for a duration of 10 minutes, whereupon the glass cylinder was saturated with ozone at a concentration of 25 g/m³. The fluidic connection to the ozone generator was removed and the glass cylinder sealed with a silicone cork.

### (c) Combination of Ozonated Sea Buckthorn Oil and Ozonated Saline

The ozonated saline water was mixed with the ozonated sea buckthorn oil in a vessel to disperse the oil as microdroplets throughout the aqueous phase. The mixture was then degassed by applying a vacuum to the vessel, affording the topical formulation base composition.

### Example 2-Peroxide Values of Sea Buckthorn Oils of Varying Degrees of Ozonation

Properties of ozonated sea buckthorn oils obtained from the protocol of Example 1 (a) were investigated. The peroxide value of each sample was determined, and the expected stable shelf-life of a topical formulation comprising the topical formulation base was calculated. The results of Example 2 are shown in Table 1 below. The predetermined duration of bubbling ozone through the sea buckthorn oil according to the protocol of Example 1 (a) is indicated in the first column of Table 4.

TABLE 4

| Duration of continuous | Peroxide | Expected stable shelf- |
|---|---|---|
| ozonation of sea | Value | life of topical |
| buckthorn oil (hours) | (mEq/kg) | formulation |
| 5 | 300-350 | 6 months |
| 8 | 380-400 | 6 months |
| 12 | 620-850 | 6 months |
| 24 | 830-1100 | 12 months |
| 48 | 1500-2200 | 12 months |

### Example 3-Properties of Ozonated Sea Buckthorn Oils Versus Non-Ozonated Sea Buckthorn Oil

Further properties of ozonated sea buckthorn oils obtained from Example 1 (a), namely those which were ozonated for 12 hours and 48 hours respectively, were investigated. Comparative measurements were performed on sea buckthorn oil which had not undergone an ozonation treatment (non-ozonated sea buckthorn oil). The results of Example 3 are shown in Table 5 below.

TABLE 5

| | Reference | | |
|---|---|---|---|
| | Sample | Sample (1) | Sample (2) |
| Appearance at room | Oil liquid | Oil liquid gel | Gel |
| temperature | | | |
| Colour | Dark orange | Orange | Orange |
| Refractive index at 20° C. | 1.460 | 1.465 | 1.478 |
| Density at 20° C. (g/cm³) | 0.91 | 0.92-0.935 | 0.92-0.94 |
| Iodine Value (iodine/100 g | 70.0 | 45.0-90.0 | 45.0-90.0 |
| oil) | | | |
| Peroxide value (mEq/kg) | 0.84 | 620-850 | 1500-2200 |
| Acid value (mg KOH/g) | 0.38 | 4.0-9.0 | 4.0-9.0 |

Reference Sample: Sea Buckthorn Seed Oil (non-ozonated)
Sample (1): Ozonated Sea Buckthorn Oil (12-hour continuous ozonation)
Sample (2): Ozonated Sea Buckthorn Oil (48-hour continuous ozonation)

### Example 4-Reduction of Total Mean Irritation (TMI)

The total mean irritation of the skin of subjects upon topical application of topical formulation base compositions was investigated.

The topical formulation base compositions which were interrogated included: a first topical formulation base composition obtained from Example 1, wherein the predetermined duration of continuous ozone treatment of the sea buckthorn oil in the protocol of Example 1 (a) was 12 hours (“Formulation Base (1)”); a second topical formulation base composition obtained from Example 1, wherein the predetermined duration of continuous ozone treatment of the sea buckthorn oil in the protocol of Example 1 (a) was 48 hours (“Formulation Base (2)”); and a reference topical formulation base comprising water, sodium chloride, and non-ozonated sea buckthorn oil in the same proportions as Formulation Base (1) and (2) (“Reference formulation base”).

Each composition was administered to the skin via topical occlusive patch over 48 hours (two patch applications, approximately 23 hours topical contact each) over three days under medical control.

The evaluation criteria of the investigation included a skin assessment based on visual assessment of erythema and of dryness. A daily irritation score for a single site was calculated according to the following formula.

$$[ erythema score from 0 to 8 ] + [ dryness score from 0 to 8 ]$$

The scoring scales shown in Tables 6 and 7 were used to determine the erythema score and the dryness score:

TABLE 6
Erythema

| Score | Description |
|---|---|
| 0 | No visible reaction |
| 1-2 | Slight redness |
| 3-4 | Moderate redness |
| 5-6 | Strong reaction, distinct redness, possibly some oedema |
| 7-8 | Very strong reaction with blistering |

TABLE 7
Dryness

| Score | Description |
|---|---|
| 0 | Skin similar to surrounding area |
| 1-2 | Skin slightly dry and crinkled |
| 3-4 | Skin markedly dry |
| 5-6 | Skin very dry, flaking |
| 7-8 | Skin very dry, severe flaking, possibly cracking |

The total mean irritation (TMI) score for a subject is the sum of the daily irritation scores for all days.

TABLE 8

| | Reference | Formulation | Formulation |
|---|---|---|---|
| | Formulation Base | Base (1) | Base (2) |
| All skin types (n = 10) | 0.3 | 0.3 | 0.3 |
| Sensitive skin (n = 8) | 0.4 | 0.4 | 0.3 |
| Rosacea skin (n = 8) | 0.4 | 0.3 | 0.3 |
| Eczema skin (n = 10) | 0.4 | 0.3 | 0.3 |

These data indicate that a topical formulation base composition comprising ozonated sea buckthorn oil that has undergone a 12-hour ozonation process (e.g. Formulation Base (1)) may be less irritating to the skin of subjects that present with rosacea or eczema compared with the Reference formulation base. These data further indicate that a topical formulation base composition comprising ozonated sea buckthorn oil that has undergone a 48-hour ozonation process (e.g. Formulation Base (2)) may be less irritating to the skin of subjects that present with sensitivity, rosacea or eczema compared with the Reference formulation base.

### Example 5-Qualitative Survey

A blind side-by-side comparative study of the Reference Formulation Base and Formulation Base (1) was conducted.

Participants were advised to apply the Reference Formulation Base (blind sample name: “Product A”) and Formulation Base (1) (blind sample name: “Product B”) in a side-by-side study. Participants were advised that Product A should be topically applied to the right-side facial cheek, and Product B should be topically applied to the left-side facial cheek. Participants were instructed to apply both Product A and Product B morning and night for 5 days.

A survey questionnaire (anonymised except for skin type data) was issued to participants on days 1, 3 and 5 of the study. All questions of the survey were asked with the below scale of evaluation: “On a scale of 1 to 10 (one being highly disagree, 10 being highly agree).”

The following questions were asked in each survey questionnaire in respect of each of Product A and Product B: “To what extent do you agree that the product improved appearance of redness?”; “To what extent do you agree that that the product calmed skin?”; “To what extent to you agree that the product moisturised skin?” and “To what extent do you agree that the product was well tolerated by your skin?”.

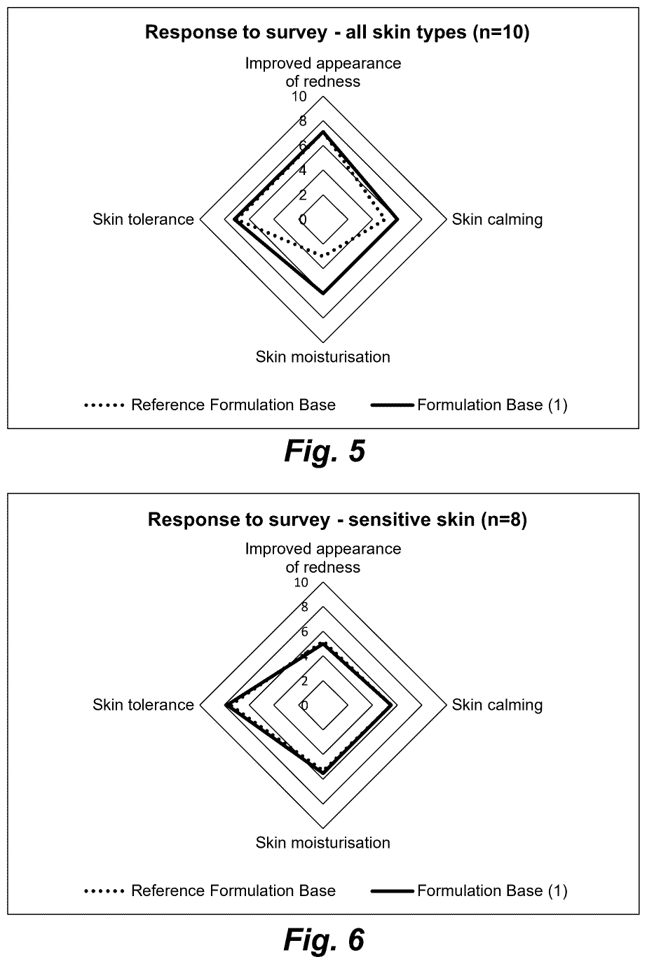

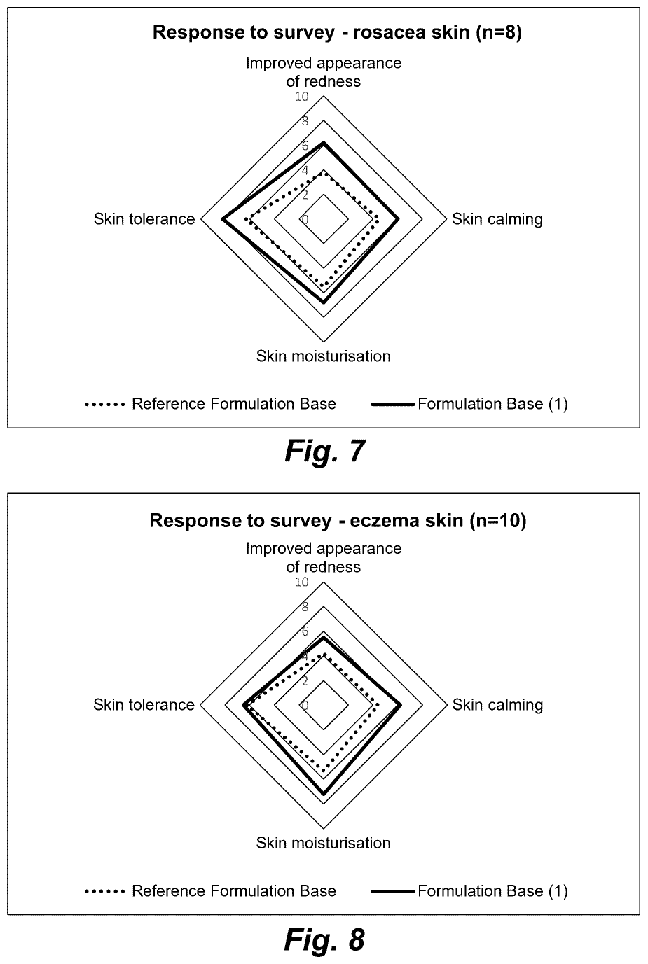

Results are presented in Tables 9 to 12 and FIGS. 5 to 8 as mean average scores across participants (separated by skin type) on a scale from 1 to 10 according to responses to the day 5 questionnaire.

TABLE 9
Question 1: To what extent do you agree that
the product improved appearance of redness?

| | “Product A” | |
|---|---|---|
| | (Reference | “Product B” |
| | Formulation Base) | (Formulation Base (1)) |
| All skin types (n = 10) | 7.1 | 7.1 |
| Sensitive skin (n = 8) | 5.2 | 5.0 |
| Rosacea skin (n = 8) | 3.8 | 6.2 |
| Eczema skin (n = 10) | 4.2 | 5.5 |

These data indicate that topical base compositions comprising ozonated sea buckthorn oil obtained from a 12-hour ozonation improve the appearance of redness of skin for subjects having rosacea and eczema compared with corresponding compositions comprising non-ozonated sea buckthorn oil.

TABLE 10
Question 2: To what extent do you
agree that the product calmed skin?

| | “Product A” | |
|---|---|---|
| | (Reference | “Product B” |
| | Formulation Base) | (Formulation Base (1)) |
| All skin types (n = 10) | 5.0 | 6.0 |
| Sensitive skin (n = 8) | 5.5 | 5.5 |
| Rosacea skin (n = 8) | 4.5 | 6.0 |
| Eczema skin (n = 10) | 4.4 | 6.2 |

These data indicate that topical base compositions comprising ozonated sea buckthorn oil obtained from a 12-hour ozonation exhibit improved skin-calming properties for subjects having rosacea and eczema compared with corresponding compositions comprising non-ozonated sea buckthorn oil.

TABLE 11
Question 3: To what extent to you agree
that the product moisturised skin?

| | “Product A” | |
|---|---|---|
| | (Reference | “Product B” |
| | Formulation Base) | (Formulation Base (1)) |
| All skin types (n = 10) | 3.0 | 6.0 |
| Sensitive skin (n = 8) | 5.3 | 5.5 |
| Rosacea skin (n = 8) | 5.5 | 6.8 |
| Eczema skin (n = 10) | 5.3 | 7.2 |

These data indicate that topical base compositions comprising ozonated sea buckthorn oil obtained from a 12-hour ozonation exhibit improved skin-moisturising properties for subjects having rosacea and eczema compared with corresponding compositions comprising non-ozonated sea buckthorn oil.

TABLE 12
Question 4: To what extent do you agree that
the product was well tolerated by your skin?

| | “Product A” | |
|---|---|---|
| | (Reference | “Product B” |
| | Formulation Base) | (Formulation Base (1)) |
| All skin types (n = 10) | 7.0 | 7.2 |
| Sensitive skin (n = 8) | 7.5 | 7.8 |
| Rosacea skin (n = 8) | 6.3 | 8.2 |
| Eczema skin (n = 10) | 6.2 | 6.5 |

These data indicate that topical base compositions comprising ozonated sea buckthorn oil obtained from a 12-hour ozonation exhibit improved skin tolerance properties for subjects across a range of skin types compared with corresponding compositions comprising non-ozonated sea buckthorn oil.

### Example 6-Topical Preservative Efficacy Test (Challenge Test)

The antimicrobial activity of the Reference Formulation Base (“Reference”) and Formulation Base (1) were investigated according to the European Pharmacopoeia standard protocol 01/2011:50103.

Antimicrobial activity was determined at inoculation (0 hours-Table 8), 6 hours after inoculation (6-hour interval-Table 9), and 48 hours after inoculation (48-hour interval-Table 10). The protocol was conducted on 250 g samples which were prepared and stored at 20 to 25° C.

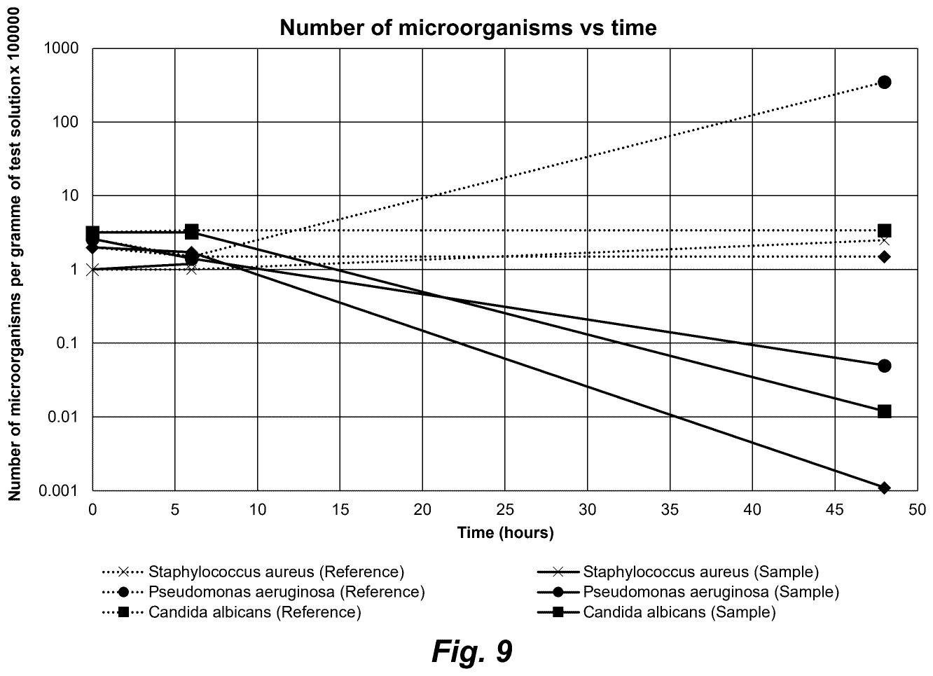

The results of these determinations are shown in Tables 13, 14, and 15, and FIG. 9 (“Sample” in FIG. 9 refers to Formulation Base (1)).

TABLE 13
Number of microorganisms - 0 hours

| | Reference | Formulation Base (1) |
|---|---|---|
| Staphylococcus aureus | 1.0 × 10⁵ | 1.0 × 10⁵ |
| Pseudomonas aeruginosa | 2.6 × 10⁵ | 2.6 × 10⁵ |
| Candida albicans | 3.2 × 10⁵ | 3.2 × 10⁵ |
| Escherichia coli | 2.0 × 10⁵ | 2.0 × 10⁵ |

TABLE 14
Number of microorganisms - 6 hours

| | Reference | Formulation Base (1) |
|---|---|---|
| Staphylococcus aureus | 1.0 × 10⁵ | 1.2 × 10⁵ |
| Pseudomonas aeruginosa | 1.5 × 10⁵ | 1.4 × 10⁵ |
| Candida albicans | 3.4 × 10⁵ | 3.2 × 10⁵ |
| Escherichia coli | 1.5 × 10⁵ | 1.7 × 10⁵ |

TABLE 15
Number of microorganisms - 48 hours

| | Reference | Formulation Base (1) |
|---|---|---|
| Staphylococcus aureus | 2.5 × 10⁵ | ND |
| Pseudomonas aeruginosa | 3.5 × 10⁷ | 5.0 × 10³ |
| Candida albicans | 3.4 × 10⁵ | 1.2 × 10³ |
| Escherichia coli | 1.5 × 10⁵ | 1.1 × 10² |

ND = no data

These data indicate that ozonated sea buckthorn oil provides an enhanced antimicrobial effect to a topical formulation base compared with non-ozonated sea buckthorn oil, and thus it may be unnecessary to include further preservatives in the topical formulation to achieve an acceptable shelf life, such as the preservatives described hereinabove. Moreover, these data indicate that the compositions, topical formulation bases, and topical formulations described hereinabove may provide a non-therapeutic antimicrobial effect.

The above embodiments are to be understood as illustrative examples of the invention. Further embodiments of the invention are envisaged. It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

### CLAUSES

1. A method for providing a topical formulation base, the method comprising:
providing ozone gas to sea buckthorn oil in a first vessel for a duration of at least 12 hours at a flow rate of at least 100 L/h to provide ozonated sea buckthorn oil having a peroxide value equal to or greater than 800 mEq/kg, wherein the providing the ozone gas comprises bubbling the ozone gas through the sea buckthorn oil;
combining deionised, purified water and sodium chloride to provide purified saline;
providing ozone gas to the purified saline in a second vessel for a duration of at least 5 minutes to provide ozonated purified saline, wherein the providing the ozone gas comprises bubbling the ozone gas through the purified saline;
combining the ozonated purified saline with the ozonated sea buckthorn oil in a third vessel, to provide a composition comprising water in an amount of from 80 to 99.8% w/w of the composition, sodium chloride in an amount of 0.05 to 5% w/w of the composition, and ozonated sea buckthorn oil in an amount of from 0.1 to 5% w/w of the composition,
wherein the combining comprises:
   providing ozone gas to a mixture of the ozonated purified saline and the ozonated sea buckthorn oil in the third vessel; wherein the providing ozone gas to the mixture comprises bubbling the ozone gas through the mixture; and
   degassing the mixture in the third vessel.
      2. The method of clause 1, wherein for at least 50% of the duration of providing the ozone gas to the sea buckthorn oil, a pressure within the first vessel is from 50 kPa to 80 kPa.
      3. The method of clause 1 or clause 2, wherein at least one of:
the ozone gas is provided to the sea buckthorn oil such that the concentration of ozone in the first vessel is at least 15 g/m³; and
the ozone gas is provided to the purified saline in a vessel until the concentration of ozone in the second vessel is at least 15 g/m³, such as at least 25 g/m³.
   4. The method of any one of clauses 1 to 3, wherein the combining comprises dispersing the ozonated sea buckthorn oil as microdroplets through the ozonated purified saline.
   5. The method of any one of clauses 1 to 4, wherein the method does not comprise combining a preservative, a fragrance, an alcohol, an emulsifier, and/or a surfactant with the water, sodium chloride, and ozonated sea buckthorn oil, wherein:
the preservative is selected from the list consisting of: benzalkonium chloride, 2-Bromo-2-nitropropane-1,3-diol, chlorhexidine digluconate, chlorphenesin, dehydroacetic acid, diazolidinyl urea, dmdm-hydantoin [1,3-bis(hydroxy-methyl)-5,5-dimethylimidazolidine-2,4-dione] ethylhexylglycerin, formaldehyde-releasing compounds (e.g, dmdm-hydantoin, quaternium-15, imidazolidinyl urea, diazolidinyl urea, and 2-bromo-2-nitropropane-1,3-diol), imidazolidinyl urea, methyldibromo glutaronitrile, methylisothiazolinone, phenoxyethanol, quaternium-15, sodium benzoate, sodium hydroxy-methylglycinate, sorbic acid, thimerosal, triclosan, and combinations thereof;
the fragrance is selected from the list consisting of: parfum, linalool, limonene, citronellol, geraniol, eugenol, rose extract (Rosa damascena), lavender oil (Lavandula angustifolia), ylang-ylang oil (Canaga odorata), and combinations thereof;
the alcohol is selected from the list consisting of ethanol and isopropyl alcohol, and combinations thereof;
the emulsifier is selected from the list consisting of: lecithin, phospholipid, and caseinate, and combinations thereof; and
the surfactant is selected from the list consisting of: polyoxyethylene (80) sorbitan monooleate, sodium lauryl sulfate, ammonium lauryl sulfate, or their ethoxylated companions, sodium laureth sulfate, and combinations thereof.
   6. The method of any one of clauses 1 to 5, wherein the combining comprises combining at least one or more further topical formulation base components with the water, sodium chloride, and ozonated sea buckthorn oil to provide the composition comprising the one or more further topical formulation base components in an amount of no more than 5% w/w of the composition, optionally wherein the one or more topical formulation base components comprises, or consists essentially of, a hydroxyethyl acrylate/sodium acryloyldimethyl taurate copolymer and/or a polyoxyethylene, polyoxypropylene block polymer.
   7. The method of any one of clauses 1 to 6 wherein, after the combining, one or more further processes are performed to provide the topical formulation base.
   8. A method for providing a topical formulation, the method comprising:
performing the method of any one of clauses 1 to 7 to provide a topical formulation base; and
combining the topical formulation base with one or more topical formulation components.
   9. The method of clause 8, wherein the topical formulation comprises the topical formulation base in an amount of at least 70% w/w of the topical formulation.
   10. The method of clause 8 or clause 9 wherein, after the combining the topical formulation base with the one or more topical formulation components, one or more further processes are performed to provide the topical formulation.
   11. A composition obtainable from the method of any one of clauses 1 to 10.
   12. A composition comprising:
ozonated deionised, purified water in an amount of 80 to 99.8% w/w of the composition; sodium chloride in an amount of 0.05 to 5% w/w of the composition; and ozonated sea buckthorn oil in an amount of 0.1 to 5% w/w of the composition;
wherein the ozonated sea buckthorn oil has a peroxide value equal to or greater than 800 mEq/kg.
   13. A topical formulation base comprising the composition of clause 12, consisting essentially of the ozonated deionised, purified water, sodium chloride, and ozonated sea buckthorn oil.
   14. A topical formulation comprising the composition of clause 12, and one or more topical formulation components in an amount of no more than 30% w/w of the topical formulation.
   15. The composition of clause 11 or clause 12, the topical formulation base of clause 13, or the topical formulation of clause 14, comprising, taken together, no more than 5% w/w a preservative, a fragrance, an alcohol, an emulsifier, and/or a surfactant, wherein:
the preservative is selected from the list consisting of: benzalkonium chloride, 2-Bromo-2-nitropropane-1,3-diol, chlorhexidine digluconate, chlorphenesin, dehydroacetic acid, diazolidinyl urea, dmdm-hydantoin [1,3-bis(hydroxy-methyl)-5,5-dimethylimidazolidine-2,4-dione] ethylhexylglycerin, formaldehyde-releasing compounds (e.g, dmdm-hydantoin, quaternium-15, imidazolidinyl urea, diazolidinyl urea, and 2-bromo-2-nitropropane-1,3-diol), imidazolidinyl urea, methyldibromo glutaronitrile, methylisothiazolinone, phenoxyethanol, quaternium-15, sodium benzoate, sodium hydroxy-methylglycinate, sorbic acid, thimerosal, triclosan, and combinations thereof;
the fragrance is selected from the list consisting of: parfum, linalool, limonene, citronellol, geraniol, eugenol, rose extract (Rosa damascena), lavender oil (Lavandula angustifolia), ylang-ylang oil (Canaga odorata), and combinations thereof;
the alcohol is selected from the list consisting of: ethanol, isopropyl alcohol, and combinations thereof;
the emulsifier is selected from the list consisting of: lecithin, phospholipid, caseinate, and combinations thereof; and
the surfactant is selected from the list consisting of: polyoxyethylene (80) sorbitan monooleate, sodium lauryl sulfate, ammonium lauryl sulfate, or their ethoxylated companions, sodium laureth sulfate, and combinations thereof.
   16. A kit comprising an applicator and the topical formulation of clause 14 or clause 15.
   17. A non-therapeutic method of providing an anti-microbial and/or anti-fungal effect to skin, the method comprising topical application of the topical formulation of clause 14 or clause 15 to a human.
   18. A therapeutic method comprising topical application of a formulation to a human, wherein the formulation comprises the composition of clause 11 or clause 12, the topical formulation base of clause 13, or the topical formulation of clause 14 or clause 15.
   19. The therapeutic method of clause 18, wherein the therapeutic method comprises preventing or reducing rosacea, eczema, erythema, or cutaneous sensory syndrome.
   20. The non-therapeutic method of clause 17 or the therapeutic method of clause 18 or clause 19, wherein the topical application comprises applying the formulation to the face of the human, preferably the periorbital region of the face.

What is claimed is:

1. A method for providing a topical formulation base, the method comprising:
providing ozone gas to sea buckthorn oil in a first vessel for a duration of at least 12 hours at a flow rate of at least 100 L/h to provide ozonated sea buckthorn oil having a peroxide value equal to or greater than 800 mEq/kg, wherein the providing the ozone gas comprises bubbling the ozone gas through the sea buckthorn oil;
combining deionised, purified water and sodium chloride to provide purified saline;
providing ozone gas to the purified saline in a second vessel for a duration of at least 5 minutes to provide ozonated purified saline, wherein the providing the ozone gas comprises bubbling the ozone gas through the purified saline;
combining the ozonated purified saline with the ozonated sea buckthorn oil in a third vessel, to provide a composition comprising water in an amount of from 80 to 99.8% w/w of the composition, sodium chloride in an amount of 0.05 to 5% w/w of the composition, and ozonated sea buckthorn oil in an amount of from 0.1 to 5% w/w of the composition,
wherein the combining comprises:
providing ozone gas to a mixture of the ozonated purified saline and the ozonated sea buckthorn oil in the third vessel; wherein the providing ozone gas to the mixture comprises bubbling the ozone gas through the mixture; and
degassing the mixture in the third vessel.

2. The method of claim 1, wherein for at least 50% of the duration of providing the ozone gas to the sea buckthorn oil, a pressure within the first vessel is from 50 kPa to 80 kPa.

3. The method of claim 1, wherein at least one of:
the ozone gas is provided to the sea buckthorn oil such that the concentration of ozone in the first vessel is at least 15 g/m³; and
the ozone gas is provided to the purified saline in a vessel until the concentration of ozone in the second vessel is at least 15 g/m³.

4. The method of claim 1, wherein the combining comprises dispersing the ozonated sea buckthorn oil as microdroplets through the ozonated purified saline.

5. The method of claim 1, wherein the method does not comprise combining a component selected from the list consisting of: a preservative, a fragrance, an alcohol, an emulsifier, a surfactant, and combinations thereof, with the water, sodium chloride, and ozonated sea buckthorn oil, wherein:
the preservative is selected from the list consisting of: benzalkonium chloride, 2-Bromo-2-nitropropane-1,3-diol, chlorhexidine digluconate, chlorphenesin, dehydroacetic acid, diazolidinyl urea, dmdm-hydantoin [1,3-bis(hydroxy-methyl)-5,5-dimethylimidazolidine-2,4-dione] ethylhexylglycerin, formaldehyde-releasing compounds (e.g, dmdm-hydantoin, quaternium-15, imidazolidinyl urea, diazolidinyl urea, and 2-bromo-2-nitropropane-1,3-diol), imidazolidinyl urea, methyldibromo glutaronitrile, methylisothiazolinone, phenoxyethanol, quaternium-15, sodium benzoate, sodium hydroxy-methylglycinate, sorbic acid, thimerosal, triclosan, and combinations thereof;
the fragrance is selected from the list consisting of: parfum, linalool, limonene, citronellol, geraniol, eugenol, rose extract (Rosa damascena), lavender oil (Lavandula angustifolia), ylang-ylang oil (Canaga odorata), and combinations thereof;
the alcohol is selected from the list consisting of ethanol and isopropyl alcohol, and combinations thereof;
the emulsifier is selected from the list consisting of: lecithin, phospholipid, and caseinate, and combinations thereof; and
the surfactant is selected from the list consisting of: polyoxyethylene (80) sorbitan monooleate, sodium lauryl sulfate, ammonium lauryl sulfate, or their ethoxylated companions, sodium laureth sulfate, and combinations thereof.

6. The method of claim 1, wherein the combining comprises combining at least one or more further topical formulation base components with the water, sodium chloride, and ozonated sea buckthorn oil to provide the composition comprising the one or more further topical formulation base components in an amount of no more than 5% w/w of the composition.

7. The method of claim 6, wherein the one or more topical formulation base components comprises a hydroxyethyl acrylate/sodium acryloyldimethyl taurate copolymer and/or a polyoxyethylene, polyoxypropylene block polymer.

8. The method of claim 1 wherein, after the combining, one or more further processes are performed to provide the topical formulation base.

9. A method for providing a topical formulation, the method comprising:
performing the method of claim 1 to provide a topical formulation base; and
combining the topical formulation base with one or more topical formulation components.

10. The method of claim 9, wherein the topical formulation comprises the topical formulation base in an amount of at least 70% w/w of the topical formulation.

11. The method of claim 9 wherein, after the combining the topical formulation base with the one or more topical formulation components, one or more further processes are performed to provide the topical formulation.

12. A composition comprising a topical formulation base, the topical formulation base comprising:
ozonated, deionised, purified water in an amount of 80 to 99.8% w/w of the topical formulation base;
sodium chloride in an amount of 0.05 to 5% w/w of the topical formulation base; and
ozonated sea buckthorn oil in an amount of 0.1 to 5% w/w of the topical formulation base;
wherein the ozonated sea buckthorn oil has a peroxide value equal to or greater than 800 mEq/kg.

13. The composition of claim 12, consisting essentially of the topical formulation base, the topical formulation base consisting essentially of the ozonated, deionised, purified water, sodium chloride, and ozonated sea buckthorn oil.

14. The composition of claim 12, further comprising one or more topical formulation components in an amount of no more than 30% w/w of the composition.

15. The composition of claim 12 comprising, taken together, no more than 5% w/w of the composition a component selected from the list consisting of: a preservative, a fragrance, an alcohol, an emulsifier, a surfactant, and combinations thereof, wherein:
the preservative is selected from the list consisting of: benzalkonium chloride, 2-Bromo-2-nitropropane-1,3-diol, chlorhexidine digluconate, chlorphenesin, dehydroacetic acid, diazolidinyl urea, dmdm-hydantoin [1,3-bis(hydroxy-methyl)-5,5-dimethylimidazolidine-2,4-dione] ethylhexylglycerin, formaldehyde-releasing compounds (e.g, dmdm-hydantoin, quaternium-15, imidazolidinyl urea, diazolidinyl urea, and 2-bromo-2-nitropropane-1,3-diol), imidazolidinyl urea, methyldibromo glutaronitrile, methylisothiazolinone, phenoxyethanol, quaternium-15, sodium benzoate, sodium hydroxy-methylglycinate, sorbic acid, thimerosal, triclosan, and combinations thereof;
the fragrance is selected from the list consisting of: parfum, linalool, limonene, citronellol, geraniol, eugenol, rose extract (Rosa damascena), lavender oil (Lavandula angustifolia), ylang-ylang oil (Canaga odorata), and combinations thereof;
the alcohol is selected from the list consisting of: ethanol, isopropyl alcohol, and combinations thereof;
the emulsifier is selected from the list consisting of: lecithin, phospholipid, caseinate, and combinations thereof; and
the surfactant is selected from the list consisting of: polyoxyethylene (80) sorbitan monooleate, sodium lauryl sulfate, ammonium lauryl sulfate, or their ethoxylated companions, sodium laureth sulfate, and combinations thereof.
